(19) United States
(12) Patent Application Publication
(10) Pub. No.: US 2013/0190207 A1
(43) Pub. Date: Jul. 25, 2013

(54) PRIORITISED GENETIC POLYMORPHISMS AND MIGRAINE SUSCEPTIBILITY

(71) Applicant: Lynette Robyn Griffiths, Queensland (AU)

(72) Inventor: Lynette Robyn Griffiths, Queensland (AU)

(73) Assignee: GRIFFITH UNIVERSITY, Nathan, Queensland (AU)

(21) Appl. No.: 13/820,449

(22) PCT Filed: Sep. 2, 2011
(86) PCT No.: PCT/AU2011/001142

§ 371 Date: Mar. 28, 2013

(30) Foreign Application Priority Data
Sep. 3, 2010 (AU) 2010903979

(51) Int. Cl. C12Q 1/68

(52) U.S. Cl. CPC C12Q 1/6883
USPC 506/9; 435/6.11

(57) ABSTRACT

The invention provides identification of an increased risk of or a predisposition to migraine according to the presence of one or more polymorphisms in the adenosine deaminase, RNA-specific, B2 (ADARB2) gene in the nucleic acid complement of a subject.

### FIELD OF THE INVENTION

THIS INVENTION relates to migraine. More particularly, this invention relates to identification of one or more polymorphisms in the adenosine deaminase, RNA-specific, B2 gene that are associated with an increased risk of migraine and uses thereof for detection of a genetic predisposition to migraine.

### BACKGROUND OF THE INVENTION

Migraine is a chronic and debilitating neurological disease which has a complex envirogenomic aetiology. It is characterised by recurrent attacks of severe headache that is usually accompanied by nausea, vomiting, photophobia, and phonophobia. The disease affects approximately 12% of the Caucasian population and females are three times more likely than males to be diagnosed (Lauver et al. 1999). Ethnic, geographic, lifestyle, and socioeconomic factors are also associated with variable risk of migraine (Lipton and Bigal 2005).

Clinical diagnosis is established by fulfilment of symptom-based criteria defined by the International Headache Society (IHS) (ICHD-II 2004), which recognises two primary sub-types: migraine with aura and migraine without aura. These subtypes have substantial symptomatic overlap, but migraine with aura sufferers experience distinguishing neurological disturbances (the aura) that usually precede the headache phase of an attack (ICHD-II, 2004).

The disorder displays strong familial aggregation, with first degree relatives of migraine probands having a 2- to 4-fold increased risk of developing the disorder compared to the general population (Cologno et al. 2003; Stewart et al. 2006). Population based twins studies report heritability estimates that range from 0.34 to 0.57 (Mulder et al. 2003; Svensson et al. 2003). A recent study of a large pedigree from a Dutch isolate reported migraine heritability estimates >0.77 (Stam et al. 2010). In general, the mode of genetic transmission of migraine is multifactorial, although autosomal dominant inheritance with reduced penetrance is evident in some affected pedigrees (Cologno et al. 2003). The migraine subtypes exhibit symptomatic heterogeneity, implying that different modifying factors may contribute to the variable expression of these clinical end-points. However, the subtypes often occur within the same individual and within the same family suggesting they have some genetic determinants in common, with possibly a major gene(s) initiating general migraine pathogenesis.

The complex genetic nature of migraine is evident from the number of loci discovered so far, which include regions on chromosome 1q31 (Lea et al. 2002), 4q21 (Björnsson et al. 2003), 4q24 (Wessman et al. 2002), 4q28 (Anttila et al. 2006), 5q21 (Nyholt et al. 2005), 6p12.2-p21.1 (Carlsson et al. 2002), 10q22-23 (Anttila et al. 2008), 11q24 (Cader et al. 2003), 14q21.2-q22.3 (Soragna et al. 2003), 15q11-q13 (Russo et al. 2005), 17p13 (Anttila et al. 2006), 18q12 (Anttila et al. 2006), 19p13 (Jones et al. 2001; Nyholt et al. 1998b), and Xq24-28 (Nyholt et al. 2000; Nyholt et al. 1998a). The predisposing gene(s) within these implicated regions have not yet been identified. However, several putative modifying genes including the Angiotensin 1 Converting Enzyme (ACE) (Lea et al. 2005), Dopamine Beta-Hydroxylase (DBH) (Fernandez et al. 2006), Dopamine 2 Receptor (DRD2) (McCarthy et al. 2001), Estrogen Receptor (ESR) (Colson et al. 2004), Insulin Receptor (INSR), 5,10-Methylenetetrahydofolate Reductase (MTHFR) (Lea et al. 2004), Serotonin Transporter (SLC6A4) (Bayerer et al. 2009), and Tumour Necrosis Factors Alpha (TNF) and Beta (LTA) (Ghosh et al. 2010) genes have been implicated through case-control association studies with some independent confirmation.

Given the complex genetic nature of migraine, there is a need to develop molecular diagnostic tests that are capable of determining whether an individual has an increased risk of or is predisposed to migraine.

### SUMMARY OF THE INVENTION

The present inventors have unexpectedly discovered new genetic polymorphisms in the adenosine deaminase, RNA-specific, B2 (ADARB2) gene that are associated with, or linked to, an increased risk of or a predisposition to migraine.

The present invention is therefore broadly directed to identification of a genetic predisposition to migraine according to the presence of one or more polymorphisms in the ADARB2 gene in the nucleic acid complement of a subject.

In a preferred form, the polymorphisms are single nucleotide polymorphisms (SNPs), and the subject is a human.

In a first aspect, the invention provides a method for identifying a subject who has an increased risk of migraine, including the step of detecting a polymorphism in the ADARB2 gene in the nucleic acid complement of the subject, wherein the presence of the polymorphism is associated with an increased risk of migraine.

In one embodiment, the subject is a human, for example, a female.

In another embodiment, the polymorphism is a single nucleotide polymorphism (SNP).

Preferably, the ADARB2 gene is human ADARB2.

Suitably, the polymorphism is a SNP at nucleotide 1230968 of human ADARB2. Alternatively, the polymorphism is a SNP at nucleotide 1227868 of human ADARB2, a SNP at nucleotide 1228206 of human ADARB2 or a SNP at nucleotide 1250184 of human ADARB2.

Preferably, the SNP at nucleotide 1230968 of human ADARB2 is an adenine to guanine change, and the change confers a threonine to alanine amino acid change in the protein encoded by human ADARB2.

In yet another embodiment, the first aspect of the invention further comprises detecting one or more additional polymorphisms in the ADARB2 gene in the nucleic acid complement of the subject, wherein the presence of the polymorphism and the one or more additional polymorphisms are associated with an increased risk of migraine.

Preferably, the ADARB2 gene is human ADARB2.

Suitably, the polymorphism is a SNP at nucleotide 1230968 of human ADARB2, and the one or more additional polymorphisms are selected from a SNP at nucleotide 1227868 of human ADARB2, a SNP at nucleotide 1228206 of human ADARB2 and/or a SNP at nucleotide 1250184 of human ADARB2.

In a second aspect, the invention provides a method for identifying a human subject who has an increased risk of migraine, including the step of detecting one or more polymorphisms in the ADARB2 gene in the nucleic acid complement of the subject, wherein the presence of the polymorphisms is associated with an increased risk of migraine.

In one embodiment, the human subject is a female subject.

In another embodiment, the one or more polymorphisms are single nucleotide polymorphisms (SNPs).

Suitably, the SNPs are selected from a SNP at nucleotide 1227868 of human ADARB2, a SNP at nucleotide 1228206 of human ADARB2, a SNP at nucleotide 1230968 of human ADARB2, and/or a SNP at nucleotide 1250184 of human ADARB2.

In a third aspect, the invention provides a kit for use in the method of the aforementioned aspects, the kit comprising one or more primers, probes and, optionally, one or more other reagents for identifying the polymorphism and/or the one or more additional polymorphisms.

In a particular embodiment, the kit comprises one or more primers for nucleic acid sequence amplification of a nucleotide sequence corresponding to at least a fragment of the ADARB2 gene.

Preferably, the ADARB2 gene is human ADARB2.

In order that the invention may be more readily understood and put into practice, one or more preferred embodiments thereof will now be described, by way of example only, with reference to the accompanying drawings.

### BRIEF DESCRIPTION OF THE DRAWINGS

FIG. 1. Manhattan Plot of autosomal genome-wide associations for migraine in the Norfolk Island pedigree. Genotype data was collected for individuals (n=285) who were selected from a core 377-member pedigree, and a pedigree-based genome-wide association study was performed by testing SNPs for association within a linkage-based probit regression model adjusted for sex and age.

FIG. 2. Haplotype block of the 4 SNPs implicated in the ADARB2 gene. Using the pGWAS strategy to assess 172 SNPs, 13 SNPs in 9 genes were prioritised, including 4 SNPs within the ADARB2 gene that made the top 0.05% cut-off. Haploview analysis showed that the 4 SNPs form a single haplotype block spanning 22 kb within the ADARB2 gene.

### DETAILED DESCRIPTION OF THE PREFERRED EMBODIMENT

Throughout this specification, unless the context requires otherwise, the words “comprise”, “comprises” and “comprising” will be understood to imply the inclusion of a stated integer or group of integers but not the exclusion of any other integer or group of integers.

The present invention is based on results of a pGWAS of the isolated population of Norfolk Island, located 1500 kilometres west of Australia. This unique population derives from a small number of British (Bounty mutineers) and Polynesian female founders forming an approximately 5700 member pedigree spanning eleven generations and exhibiting substantial inbreeding and admixture.

Three hundred seventy-seven founder-related adults were phenotyped for migraine using the diagnostic criteria of the International Headache Society, and 285 of these individuals were genotyped. Association results were adjusted for sex, age, admixture, and inbreeding and SNPs were prioritised based on both statistical and biological significance. Statistical significance was based on the top 0.05% of SNPs ranked by P-value and biological significance was assessed based on published annotation data and knowledge of disease pathology.

Ninety-six migraine affected individuals in the Norfolk pedigree were identified, yielding a point prevalence estimate of 25.5%. Pedigree analysis indicated that the migraine phenotype had strong heritability (h²=0.53, P=0.016). Pedigree-based genome-wide association study analysis and SNP prioritisation incorporating biological annotation implicated thirteen SNPs in nine genes as being associated with migraine risk at the gene-wide level. Subsequent SNP prioritisation incorporating biological annotation implicated four SNPs forming a 22 kb haplotype block within the ADARB2 gene as being associated with migraine risk.

The present invention therefore has arisen from the identification of a genetic predisposition to migraine according to the presence of a polymorphism in the ADARB2 gene in the nucleic acid complement of a subject.

Typically, the polymorphism is a SNP.

The term “single nucleotide polymorphism” is used herein to indicate any nucleotide sequence variation in an allelic form of a gene that occurs in a subject (e.g., human) population. This term encompasses alternative nucleotides, mutation, insertion, deletion, and other like terms that indicate specific types of SNPs.

Preferably, the subject is a human, including both males and females.

Thus, in one aspect, the invention provides a method for identifying a subject (e.g., a human) who has an increased risk of migraine, including the step of detecting a polymorphism in the ADARB2 gene in the nucleic acid complement of the subject, wherein the presence of the polymorphism is associated with an increased risk of migraine.

By an “increased risk” of migraine is meant a subject that is identified as having a higher than normal chance of developing a migraine, compared to the general population. Subjects with an increased risk of migraine may be considered to be predisposed to, or have a predisposition to, migraine. As used herein, “predisposed” and “predisposition”, in the context of migraine, mean that an individual is susceptible to, or has an increased likelihood or probability of, suffering from migraine, and includes situations where the individual is not yet exhibiting clinical symptoms of migraine as well as where the individual is displaying symptoms of migraine.

As used herein, “migraine” includes migraine with aura (MA) and migraine without aura (MO).

The term “polymorphism” (terms such as “polymorphism”, “mutation”, “mutant”, “variation”, and “variant” are used herein interchangeably) refers to a difference in a DNA or RNA sequence or sequences among individuals, groups or populations, which give rise to a statistically significant phenotype or physiological condition. Examples of genetic polymorphisms include mutations that result by chance or are induced by external features. A polymorphism may be indicative of a disease or disorder, and/or a predisposition to a disease or disorder. In a preferred aspect, the polymorphisms of the present invention are indicative of an increased risk of, including a predisposition to, migraine.

In one embodiment, the polymorphism is a SNP.

A genetic locus comprising the ADARB2 gene may be referred to as a “gene”, a “nucleic acid”, a “locus”, a “genetic locus”, or a “polynucleotide”. Each refers to a polynucleotide, which includes the gene's 5′- and 3′-terminal regions, promoter, introns, and exons. Accordingly, the ADARB2 gene of the present invention is intended to include coding sequences, intervening sequences and regulatory elements controlling transcription and/or translation. A genetic locus is intended to include all allelic variations of the DNA sequence on either or both chromosomes. Consequently, homozygous and heterozygous variations of the instant genetic loci are contemplated herein.

The term “nucleic acid” as used herein designates single- or double-stranded mRNA, RNA, cRNA, and DNA (inclusive of cDNA and genomic DNA), and DNA-RNA hybrids.

The term “nucleic acid complement” of a subject refers to the total nucleic acid content of a subject (e.g., as found in a biological sample, such as a cell, of a subject), and includes the full set of genes (i.e., DNA), their translation products (i.e., RNA) and the non-coding genetic material in a subject.

Preferably, the ADARB2 gene is human. ADARB2.

Typically, the polymorphism is a SNP at nucleotide 1230968 of human ADARB2. Alternatively, the polymorphism is a SNP at nucleotide 1227868 of human ADARB2, a SNP at nucleotide 1228206 of human ADARB2 or a SNP at nucleotide 1250184 of human ADARB2.

Preferably, the SNP at nucleotide 1230968 of human ADARB2 is an adenine to guanine change, and the change confers a threonine to alanine amino acid change in the protein encoded by human ADARB2.

In another embodiment, this aspect of the invention further comprises detecting one or more (e.g., 2, 3, 4, 5, 6, 7, 8, 9, 10, 15, 20, 25, 30, 35, 40, 45, 50, 60, 70, 80, 90, or greater) additional polymorphisms in the ADARB2 gene in the nucleic acid complement of the subject, wherein the presence of the polymorphism and the one or more additional polymorphisms are associated with an increased risk of migraine.

Preferably, the ADARB2 gene is human ADARB2.

Typically, the polymorphism is a SNP at nucleotide 1230968 of human ADARB2, and the one or more additional polymorphisms are selected from a SNP at nucleotide 1227868 of human ADARB2, a SNP at nucleotide 1228206 of human ADARB2 and/or a SNP at nucleotide 1250184 of human ADARB2.

Also contemplated by the invention is a method for identifying a subject (e.g., a human) who has an increased risk of migraine, including the step of detecting one or more (e.g., 2, 3, 4, 5, 6, 7, 8, 9, 10, 15, 20, 25, 30, 35, 40, 45, 50, 60, 70, 80, 90, or greater) polymorphisms in the ADARB2 gene in the nucleic acid complement of the subject, wherein the presence of the polymorphisms is associated with an increased risk of migraine.

Preferably, the ADARB2 gene is human ADARB2.

The methods disclosed herein may be used independently of clinical diagnosis, or may be used in conjunction therewith to confirm or assist clinical diagnosis of migraine, inclusive of migraine with aura and migraine without aura.

Furthermore, the methods of the invention may be used in combination with additional methods that identify other genetic polymorphisms associated with migraine.

Generally, the methods of the invention are nucleic acid-based methods that include an analysis of the nucleic acid complement of the subject.

Nucleic acid-based methods may include the step of obtaining an isolated nucleic acid sample from the subject.

For the purposes of this invention, by “isolated” is meant material that has been removed from its natural state or otherwise been subjected to human manipulation. Isolated material (e.g., nucleic acid) may be substantially or essentially free from components that normally accompany it in its natural state, or may be manipulated so as to be in an artificial state together with components that normally accompany it in its natural state. Isolated material may be in native or recombinant form.

An isolated nucleic acid sample corresponding to the nucleic acid complement of the subject may be obtained from any appropriate subject source of nucleic acid, such as lymphocytes or any other nucleated cell type, preferably obtainable by a minimally-invasive method.

The isolated nucleic acid may be in the form of genomic DNA, RNA or cDNA reverse-transcribed from isolated RNA.

In a particular embodiment of the invention, an isolated nucleic acid sample corresponding to the nucleic acid complement of the subject may be a product of nucleic acid sequence amplification.

It will be readily appreciated by persons skilled in the art that the ADARB2 gene may be used as the basis for designing primers that allow amplification of at least a fragment of the ADARB2 gene.

In this regard, it will be appreciated that preferred diagnostic methods employ a nucleic acid sequence amplification technique.

Suitable nucleic acid amplification techniques are well known in the art, and include polymerase chain reaction (PCR) and ligase chain reaction (LCR) as, for example, described in Chapter 15 of CURRENT PROTOCOLS IN MOLECULAR BIOLOGY (Eds. Ausubel et al. John Wiley & Sons NY, 2000); strand displacement amplification (SDA) as, for example, described in U.S. Pat. No. 5,422,252; rolling circle replication (RCR) as, for example, described in Liu et al. (1996, J. Am. Chem. Soc. 118:1587), International application WO 92/01813 and International Application WO 97/19193; nucleic acid sequence-based amplification (NASBA) as, for example, described by Sooknanan et al. (1994, Biotechniques 17:1077); ligase chain reaction (LCR) as, for example, described in International Application WO89/09385; Q-β replicase amplification as, for example, described by Tyagi et al. (1996, Proc. Natl. Acad. Sci. USA 93:5395); and helicase-dependent amplification as, for example, described in International Publication WO 2004/02025.

As used herein, an “amplification product” is a nucleic acid produced by a nucleic acid sequence amplification technique.

A preferred nucleic acid sequence amplification technique is PCR.

Notwithstanding the foregoing, the invention contemplates other nucleic acid detection methods that may be useful for detecting a SNP polymorphism in the ADARB2 gene.

For example, a PCR method that may also be useful is Bi-PASA (Bidirectional PCR Amplification of Specific Alleles), as for example described in Liu et al. (1997, Genome Res. 7:389-399).

Another potentially useful PCR method as allele-specification oligonucleotide hybridization, as for example described in Aitken et al. (1999, J. Natl. Cancer Inst. 91:446-452).

The SNPs according to the invention may be identified by direct sequencing of a PCR amplification product, for example. An example of nucleic acid sequencing technology is provided in Chapter 7 of CURRENT PROTOCOLS IN MOLECULAR BIOLOGY (Eds. Ausubel et al. John Wiley & Sons NY, 1995-2001).

In yet another embodiment, mass spectroscopy (such as MALDI-TOF) may be used to identify nucleic acid polymorphisms according to mass. In a preferred form, such methods employ mass spectroscopic analysis of primer extension products, such as using the MassARRAY™ technology of Sequenom.

In a further embodiment, a SNP of the invention may be identified by a microarray.

Microarray technology has become well known in the art and examples of methods applicable to microarray technology are provided in Chapter 22 of CURRENT PROTOCOLS IN MOLECULAR BIOLOGY (Eds. Ausubel et al. John Wiley & Sons NY, 1995-2001).

With respect to the present invention, a preferred microarray format comprises a substrate such as a glass slide or chip having an immobilized, ordered grid (e.g., individually addressable) of a plurality of nucleic acid molecules, such as cDNA molecules, although without limitation thereto.

A microarray would typically comprise one or more nucleic acids having one or more polymorphisms in the ADARB2 gene, as disclosed herein, together with control nucleic acids.

Such a microarray could also include a plurality of other nucleic acids indicative of other diseases that have an underlying genetic basis and be useful in large scale genetic screening, for example.

It will also be appreciated that the methods of the invention also extend to a methods of analysis of one or more gene sequence databases to identify an individual or individuals having one or more polymorphisms in the ADARB2 gene, as herein described.

In this regard, an increasing aspect of molecular medicine is the establishment of computer-searchable databases that comprise genetic information obtained from patients, which databases may readily be interrogated to correlate the presence of one or more polymorphisms in the ADARB2 gene, as herein described, with genetic information obtained from a particular patient.

It will also be appreciated from the foregoing that the invention contemplates a kit for use in the methods described herein, the kit including one or more primers, probes and, optionally, one or more other reagents for identifying one or more polymorphisms in the ADARB2 gene.

A “primer” is usually a single-stranded oligonucleotide, preferably having 15-50 contiguous nucleotides, which is capable of annealing to a complementary nucleic acid “template” and being extended in a template-dependent fashion by the action of a DNA polymerase, such as Taq polymerase, RNA-dependent DNA polymerase or Sequenase™ It will be readily appreciated by persons skilled in the art that the ADARB2 gene may be used as the basis for designing primers that allow amplification of at least a fragment of the ADARB2 gene.

Thus, in a particular embodiment of the invention, a fragment may be a product of nucleic acid sequence amplification.

A “probe” may be a single or double-stranded oligonucleotide or polynucleotide, suitably labelled for the purpose of detecting complementary sequences in Northern or Southern blotting, for example.

One or more other reagents are contemplated, such as detection reagents useful in enzymatic, colorimetric and/or radionuclide-based detection of nucleic acids, although without limitation thereto.

So that the present invention may be more readily understood and put into practical effect, the skilled person is referred to the following non-limiting examples.

### EXAMPLES

### Experimental Procedures

### Sample Ascertainment

The study protocol was approved by Griffith University Human Research Ethics Committee. All subjects provided signed, informed consent prior to participation. Data collection procedures have been described in detail elsewhere (Bellis et al. 2005). In brief, subjects were ascertained based on permanent resident status (not selected on phenotypes of interest), to ensure sampling of individuals from the same genealogical background. Phenotypic data and biological specimens were obtained from 600 subjects (261 males, 339 females) with a mean age of 50.8 years (standard deviation of 16.4 years). Venous blood specimens were available for 600 individuals from their visit to a temporary research clinic on Norfolk Island, carried out during 2000. Blood samples were collected in an EDTA tube. DNA was isolated from a 10-20 mL sample using a standard salting-out procedure (Miller et al. 1988). DNA concentration (ng/μl) and purity (260 nm:280 nm) were determined spectrophometrically using the NanoDrop ND-1000 (NanoDrop Technologies, Inc.). Genealogical data was obtained via questionnaire, and municipal and historical records. Phenotypic data was obtained via a comprehensive medical questionnaire that included a section specific to migraine. Detailed questions regarding family history, symptoms, triggers, and medication was obtained. Migraine diagnosis was established in accordance with current IHS guidelines (ICHD-II 2004).

### Genealogical Structure

Historical and genealogical records indicate Pitcairn Island was settled by 9 Isle of Man Bounty mutineers, 12 Tahitian women and 6 Tahitian men in 1790 (Hoare 1999). The Pitcairn Islanders were resettled on Norfolk Island in the mid-19^{th} century. Pedigree reconstruction and validation has confirmed current descendents possess lineages to all 9 Bounty mutineers, 6 of the Tahitian women and 2 additional Caucasian sailors who joined the small colony during the early 19^{th} century (Bellis et al. 2005; Macgregor et al. 2010; McEvoy et al. 2009). A total of 377 individuals were determined to have familial links to these 17 founders and were integrated into all heritability and association analyses. The size and complexity of the genealogical structure (N=6,537) and large volume of missing data prohibited direct use in variance component linkage analysis (Bellis et al. 2008). To facilitate analysis, the pedigree was split (N=1,078) using a peeling algorithm in the pedigree database management system PEDSYS (Southwest Foundation for Biomedical Research, San Antonio, Texas, USA) (Dyke 1996). This 1,078 member pedigree has been previously employed in genome-wide screens of cardiovascular risk traits (Bells et al. 2008).

### SNP Genotyping

DNA samples were genotyped according to the manufacturer's instructions on Illumina Infinium High Density (HD) Human610-Quad DNA analysis BeadChip version 1. A total of 620,901 genome-wide markers were genotyped in a sub-sample of 285 related individuals (135 males; 150 females). These related individuals include 76 migraine cases (22 males; 54 females). Markers had a median spacing of 2.7 kb (mean=4.7 kb) throughout the genome. Each Human610-Quad DNA analysis BeadChip employed a four-sample format requiring 200 ng of DNA per sample. Samples were scanned on the Illumina BeadArray 500GX Reader. Raw data was obtained using Illumina BeadScan image data acquisition software (version 2.3.0.13). Preliminary analysis of raw data was undertaken in Illumina BeadStudio software (version 1.5.0.34) with the recommended parameters for the Infinium assay, and using genotype cluster files provided by Illumina.

Individuals with a call rate below 95% and SNPs with a call rate below 99%, deviating from Hardy-Wienberg equilibrium (p_{HWE}<1×10⁻⁷) or with a minor allele frequency of less than I % were excluded from analysis. Genotypic data was analysed for discrepancies, including mendelian inheritance violations using the PEDSYS program INFER (Dyke 1996) and Simwalk2 (Sobel et al. 2002). The Pedigree RElationship Statistical Test (PREST) was used to verify the pedigree structure and detect relationship misspecification (McPeek and Sun 2000). Discrepant genotypes were blanked prior to analysis. SNPs were annotated using information available from the National Centre for Biotechnology Information (NCBI), Build 36.3.

### Statistical Analysis: Heritability and Association

General characteristics of the subjects in each group were assessed using SPSS version 14.0 for windows (SPSS, Chicago, Ill.). All statistical analyses on related individuals were conducted using variance components-based methodology implemented in the Sequential Oligonucleotide Linkage Analysis Routines (SOLAR) version 4.0.6 software package (Almasy and Blangero 1998) (Southwest Foundation for Biomedical Research, San Antonio, Tex., USA). Heritability (h²) estimates were calculated as the ratio of the trait variance that is explained by additive polygenic effects to total phenotypic variance of the trait (Göring et al. 2001). The applied polygenic model assumes an infinite number of genetic factors, each with a small additive effect contributing to the trait variance (“narrow sense” heritability). Estimates were screened for the covariate effects of age, age-squared, sex, and their interactions to allow for differential symptom prevalence in males and females and adjust for the variable age of onset. Covariates with p-values less than or equal to 0.05 were retained in the final model. Dichotomous trait analysis was enabled by assuming a liability threshold model, with an underlying multivariate normal distribution (Duggirala et al. 1997).

Two additional covariates, of potential interest to this study, the inbreeding (F) and ancestry coefficient (Q) were also screened. The ancestry coefficient is a measure of the degree of Polynesian and Caucasian admixture in the Norfolk pedigree. A value of 0 indicates no Polynesian ancestry. A value of 1 signifies full Polynesian ancestry. A significant ancestry-specific effect would warrant further investigation by admixture mapping. In contrast, F reflects the probability that 2 alleles at a locus are identical by descent (IBD). A value of 0 indicates no inbreeding. As the coefficient approaches 1 the level of inbreeding increases. A significant covariate effect would support recessive inheritance and founder effect for migraine.

Both these coefficients have been previously described in the Norfolk population (Macgregor et al. 2009). Briefly, coefficients were calculated using PEDIG software assuming the complete founder pedigree that spans more than 200 years and includes the direct descendents of the population founders as well as recent married-in individuals (Boichard 2002; Macgregor et al. 2009). Specifically, the Meuwissen and Luo method was used to calculate F (Meuwissen and Luo 1992). The covariate effects of ancestry and inbreeding were explored by mixed (polygenic) model analysis

Genome-wide association testing was performed using measured genotype analysis (Boerwinkle et al. 1986), embedded in a variance components-based linkage model (Blangero et al. 2005). This assumed an additive model of allelic effect, where SNP genotypes AA, AB and BB were coded as −1, 0 and 1, respectively, and used as a linear predictor of phenotype (Blangero et al. 2005). A total of 544,590 SNPs across chromosomes 1 to 22 were available for analysis. Genome-wide significance of a genetic locus was based on a local type I error of a=0.05/544,590 SNPs, which equals 9.2×10⁻⁸ by Bonferroni adjustment.

Single nucleotide polymorphism results were annotated using the Whole Genome Association Study Viewer (WAGViewer) program (http://people.genome.duke.edu/˜dg48/WGAViewer/) (Ge et al. 2008) and NCBI Build 37.1.

### Results

### Migraine Prevalence and Heritability Estimation

In total, we analysed migraine phenotype information from a 377-member pedigree previously described (Bellis et al, 2008 and Macgregor et al, 2009). Of this pedigree, 96 individuals screened positive for migraine according to the IHS criteria and our research Neurologist. This yields a migraine prevalence estimate of 25.5% for Norfolk Island which is approximately twice as high as the established prevalence of 12% in outbred Caucasian populations (Lipton et al. 2007). This strong familial clustering is consistent with the notion that inherited factors play a role in disease risk and establishes the Norfolk Island population as “high risk” for migraine. Sub-classification of the migraine phenotype indicated 64 (66.7%) of these subjects had MA and 32 (33.3%) had MO. The remaining 281 individuals were negative for migraine.

The demographic and familial characteristics of migraine cases and unaffecteds in the 377-member pedigree were assessed. A high proportion of affected females were observed (74%), which is consistent with the female-male ratio of approximately 3 to 1 (Launer et al. 1999). Migraineurs were slightly younger on average compared to non-migraineurs (46 vs 50 yrs). Migraine sufferers had slightly higher Polynesian ancestry values (Q) compared to non-migraineurs (0.114 vs 0.108, P>0.1). Of the 377 individuals, 60 displayed some level of inbreeding (mean F=0.026). However, when all 377 subjects, including recent married-in individuals were considered, inbreeding was relatively modest (0.0042) and the coefficient was heavily skewed towards zero. Migraine sufferers showed slightly lower average levels of inbreeding (F) than non-migraineurs (0.0034 vs 0.0044, P>0.1). Mixed model analysis confirmed that Polynesian admixture and inbreeding were not significant predictors of migraine (P>0.1). SOLAR analysis estimated heritability of the migraine phenotype after adjusting for sex as 0.53 (SE=0.302; P=0.016), which is consistent with other studies. A heritability of 0.53 implies at least half of the variation in migraine risk is influenced by genetically inherited factors and warrants the conducting of a genome-wide association study.

### Pedigree-Based Genome-Wide Association Study

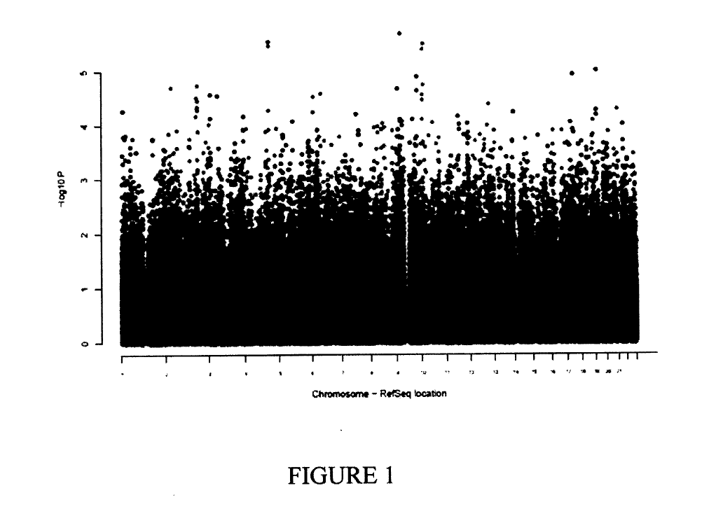

Illumina 610-quad genotype data was collected for n=285 individuals who were selected from the core 377-member pedigree and were highly informative for linkage. A pGWAS was performed by testing SNPs for association within a linkage-based probit regression model adjusted for sex and age. A Manhattan plot of P-values is depicted in FIG. 1.

We also prioritised SNPs based on their potential relevance to disease pathophysiology (Igl et al. 2010). This approach prioritises SNPs by P-value as well as evidence for a functional role in disease pathology. Specifically, we focused on the top 0.05% of SNPs with the lowest P-value. The results of our pGWAS revealed 172 SNPs in this region of the probability distribution. We then assessed these SNPs according to whether they were physically near genes with known annotation, placing more value on genes with a putative role in neurology and/or migraine pathology (i.e., candidate genes). Specifically, genes that are known to a) be expressed in the brain or central nervous system (CNS), b) regulate neurological pathways (e.g., neurotransmitters) and/or c) be plausible related to migraine neuropathology (e.g., cellular hyperexcitability; ion channel disruption).

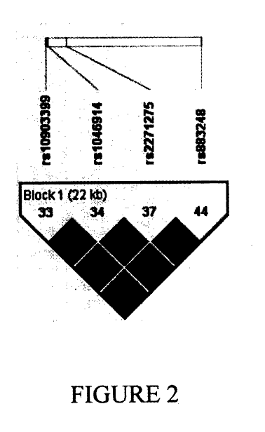

Using this strategy to assess the 172 SNPs, we prioritised 13 SNPs in 9 genes. In particular, we found the ADARB2 gene to be of the most interest, statistically and functionally. The ADARB2 gene is expressed in the central nervous system and is involved in RNA editing and downstream regulation of neurotransmitters (Maas et al. 2003). Neurotransmitter pathways are known to be disrupted in the rare MA subtype FHM (De Fusco et al. 2003; Dichgans et al. 2005; Ophoff et al. 1996). Interestingly, there were 4 SNPs within the ADARB2 gene that made the top 0.05% cut-off (Table 1). Further examination showed a total of 245 SNPs were typed across the gene. This means that even considering the overly stringent Bonferroni adjusted probability threshold, these 4 SNPs were statistically significant at the gene-wide level (P<2.0×10⁻⁴). Haploview analysis showed the 4 SNPs form a single haplotype block spanning 22kb within the ADARB2 gene (FIG. 2). Interestingly, one of the ADARB2 SNPs (rs2271275) confers a Thr to Ala amino acid change, providing a plausible candidate variant for involvement in disease causation. Risk analysis indicated that the Thr variant of the rs2271275 SNP produces an OR of 2.01, implying an approx 2-fold increased risk of migraine in carriers of this allele.

### Discussion

This study was conducted to assess the genetic risk of migraine in a genetic isolate from Norfolk Island in the South Pacific. The Norfolk Island population is of particular interest for gene-mapping because of its large, well-documented pedigree structure, genetic founder and admixture effects, as well as extreme cultural and geographical isolation from mainland Australia. We identified a 377-member pedigree within the Norfolk Island population that was connected to the founders and available for phenotyping and genotyping. Individuals were diagnosed following strict IHS 2004 guidelines and an elevated rate of migraine in the pedigree was observed (25%), which is relatively high compared to estimates in general outbred populations of similar ethnicity. Whilst there is no accurate data for mainland Australia, recent data from the American Migraine Prevalence and Prevention (AMPP) study estimated total migraine prevalence at 11.7% (males 5.6%; females 17.1%) in 162,576 participants (Lipton et al. 2007). Similar estimates are reported in the results from global meta-analysis of epidemiological studies (Stovner et al. 2007). One year migraine prevalence in adults was estimated as 11%, while Global lifetime prevalence was slightly higher at 14%.

The migraine subtypes (MA and MO) exhibit symptomatic heterogeneity implying that different modifying factors may contribute to the variable expression of these clinical end-points. However, in this pedigree the subtypes often occur within the same individual and within the same family, suggesting they have some genetic determinants in common with possibly a major gene(s) initiating general migraine pathogenesis. Subtype analysis was not performed for this reason, and because of a reduction in effective sample size and consequent statistical power. Heritability analysis of the migraine phenotype showed it was under the influence of considerable additive genetic effects. Heritability was estimated at 0.53, which is similar to reports of population-based twin studies (Mulder et al. 2003).

Once prevalence and heritability was established for the pedigree, we conducted a pGWAS using a statistical and biological significance approach. Using this prioritisation method, the pGWAS results implicated 4 SNPs forming a 22 kb haplotype block within the ADARB2 gene as being associated with migraine risk. Interestingly, one of the ADARB2 SNPs confers a Thr to Ala amino acid change, providing a plausible candidate variant for involvement in disease causation. This non-synonymous variant, rs2271275, has not previously been implicated directly in migraine susceptibility, but has previously been associated with Early-Onset Obsessive-Compulsive Disorder [MIM 164230] in American families (Hanna et al. 2007).

The human RNA-editing deaminase-2 (ADARB2) gene, located on chromosome 10p15, was first characterised in 1997 (Mittaz et al. 1997). ADARB2 is a member of the double-stranded RNA (dsRNA)-specific adenosine deaminase gene family of RNA-editing enzymes. In particular these genes edit transcripts expressed in the CNS (Keegan et al. 2004). RNA editing alters RNA sequences encoded by DNA. By altering one or two SNPs in the RNA sequence, RNA editing may alter the codon, create a splice site or alter the RNA structure of the target RNA sequence. Genes in this family catalyze the deamination of adenosine to create inosine, which is translated as a guanosine. These enzymes have the potential to change the primary sequence information in an RNA sequence, altering the codon meaning so that more than one protein isoform can be synthesized from a single gene (Bass 2002).

RNA editing plays a crucial role in the expression of certain gene products. The editing may change the sequence of mRNAs, resulting in the synthesis of proteins not encoded by the original gene sequence (Smith et al. 1997). This may lead to severe disorders, as is seen in amyotrophic lateral sclerosis (ALS) (MIM 105400). In affected individuals editing of the messenger RNA encoding the GluR2 subunit of glutamate AMPA receptors in the spinal motor neurons is defective (Kawahara et al. 2004). RNA editing in ALS-affected individuals fails to substitute an arginine for a glutamine residue at a crucial site in the GIuR2 subunit. This interferes with normal functioning of the glutamate receptors and may be a contributory cause of neuronal death in ALS patients. Hypothetical roles for RNA-editing genes have been suggested for complex neurological disorders such as epilepsy, depression and schizophrenia, particularly where past studies implicate altered levels of glutamate and serotonin (Maas et al. 2006).

This dsRNA-specific adenosine deaminase gene family of RNA-editing enzyme genes and their substrates display high levels of expression in CNS, particularly the brain (Paul and Bass 1998). The human homologue of ADARB2 in the rat, RED2, displays brain-specific expression (Melcher et al. 1996). In situ hybridization in rat brain revealed differential expression of RED2 mRNA throughout the brain with high transcript levels occurring in the olfactory bulb and thalamus. Two RNA-editing substrates modified by adenosine deamination and of interest to this study are glutamate and serotonin receptor gene RNAs (Maas et al. 2003).

Glutamate and Serotonin are major excitatory neurotransmitters in the mammalian CNS and are implicated in migraine pathophysiology. Data from human and animal migraine studies indicate' glutamate modulates CNS sensitization, which activates the trigeminal system and propagates migraine attacks (Vikelis and Mitsikostas 2007). The trigeminovascular nociceptive pathway can also be activated by altered serotonergic neurotransmission, resulting in migraine (Hamel and Currents 2007). Selective serotonin 5-HT1B/1D agonists have been used extensively in the treatment of migraine attacks (Ferrari et al. 2001). The role of the serotonergic pathway in migraine is further supported by molecular genetic studies that report positive associations between polymorphisms in the serotonin transporter gene, SLC6A4 [MIM182138] and migraine (Park et al. 2007; Racchi et al. 2004; Johnson and Griffith 2005; Bayerer et al. 2010; Juhasz et al. 2003; Marziniak et al. 2005; Yilmaz et al. 2001; Ogilvie et al. 1998).

Glutamate-gated receptor channels (GluR) mediate fast excitatory neurotransmission in the mammalian brain. Adenosine deamination by RNA editing occurs in GIuR pre-mRNA transcripts, which may act to diversify the mammalian genome (Seeburg 1996; Seeburg et al. 1998). Modification of glutamate receptor B (GluR) B pre-mRNA by RNA-editing has been demonstrated in rat models (Melcher et al. 1996). In addition to GIuR, serotonin subtype 2C receptor (5-HTR2C) with altered G protein-coupling efficacy are generated by adenosine deaminase RNA editing (Maas et al. 2003; Wang et al. 2000).

Migraine pathophysiology suggests a role of serotonergic and/or glutamatergic pathways in disease aeitiology. Furthermore, variants in genes belonging to serotonergic pathways have been implicated in migraine. Specifically, positive associations within the serotonin transporter gene (SLC6A4; M1M182138) on chromosome 17q11.1-q12 (Bayerer et al. 2009; Johnson and Griffiths 2005; Juhasz et al. 2003; Marziniak et al. 2005; Ogilvie et al. 1998; Park et al. 2006; Racchi et al. 2004; Yilmaz et al. 2001) and the serotonin (5-hydroxytryptamine; 5-HT) receptor 2C gene (HTR2C; MIM312861) on chromosome Xq24 (Kusumi et al. 2004). A recent study evaluating 19 serotonin-related genes and migraine susceptibility in a Spanish population implicated the serotonin 5-HT-2B receptor (HTR2B; MIM 601122; 2q36.3-q37.1) and monoamine oxidase A (MAOA; MIM 309850; Xp11.23) genes in MO, and dopa decarboxylase (DDC; MIM 107930; 7p11) gene in MA (Corominas et al. 2009). Negative association with SLC6A4 and HRT2C are also reported (Oterino et al. 2007; Todt et al. 2006). The varying results may reflect differences in study design, sample size, ethnic background or may indicate that different genes in the serotonergic pathway are involved in different populations.

Whilst candidate gene studies have yet to implicate genes belonging to glutamatergic pathways, the results of clinical drug trials provide strong support for involvement Anticonvulsants such as topiramatea, valproatea, gabapenti, and lamotrigine decrease glutamate levels and enhance gamma-amino butyric acid (GABA) (Silberstein 2006). Migraine with aura is believed to be the result of cortical spreading depression (CSD), a wave of excitation followed by depression that travels across the cerebral cortex at 2-3 mm/min. One feature of CSD, particularly relevant to our finding, is that it causes transient increases in glutamate, which drugs such as anitconvulsants act to prevent (Goadsby et al. 2009; Olesen et al. 1990; Pietrobon and Striessnig 2003). Glutamate receptors are currently being targeted by emerging migraine therapeutics, such as the preventative drug, ADX10059, a mGluR5 modulator (Sprenger and Goadsby 2009).

Recently, photon emission computed tomography images and MRI-scans in migraineurs and control demonstrated, for the first time, a significant increase of brainstem SERT-availability in migraineurs (Schuh-Hofer et al. 2007). Results suggested migraine may result in dysregulation of the brainstem serotonergic system. Serotonin pathways have long been targeted by pharmaceuticals. Serotonin antagonists were introduced as a migraine therapy, as the disorder was believed to result from excess of this excitatory neurotransmitter (Silberstein 2006). Antidepressants, such as the selective serotonin reuptake inhibitors (SSRIs), have been used to treat migraine, but are not commonly used today (Silberstein 2006). Valproic acid, another migraine drug interacts with the central serotonin system (Moskowitz 1992).

The RNA-editing enzyme detected in our pGWAS may explain the involvement of serotonergic and glutamatergic system disruption in migraine pathophysiology via post-transcriptional modifications.

Throughout this specification, the aim has been to describe the preferred embodiments of the invention without limiting the invention to any one embodiment or specific collection of features. Various changes and modifications may be made to the embodiments described and illustrated herein without departing from the broad spirit and scope of the invention.

All computer programs, algorithms, patent and scientific literature referred to in this specification is incorporated herein by reference in their entirety.

TABLE 1
ADARB2 SNPs (n = 4) selected from association results from the Norfolk Cohort

| | | | | | | | DIST. | | | | |
|---|---|---|---|---|---|---|---|---|---|---|---|
| | | | | | MINOR/ | | TO | | | | |
| SNP REF. | | | POSITION | | MAJOR | | GENE | | GENE | | |
| NO. | P-VALUE | BETA | (BP) | FUNCTION | ALLELE | MAF | (BP) | GENE | ID | MIM | LOCATION |
| rs10903399 | 7.68E−05 | 0.6377 | 1227868 | DOWNSTREAM | C/T | 0.33063 | 205 | ADARB2 | 105 | 602065 | 10p15.3 |
| rs1046914 | 3.43E−05 | 0.6728 | 1228206 | 3 PRIME UTR | G/A | 0.327739 | 0 | ADARB2 | 105 | 602065 | 10p15.3 |
| rs2271275 | 2.67E−05 | 0.6495 | 1230968 | NON-SYNON | G/A | 0.367661 | 0 | ADARB2 | 105 | 602065 | 10p15.3 |
| rs883248 | 3.83E−06 | 0.6656 | 1250184 | INTRONIC | G/A | 0.438962 | 0 | ADARB2 | 105 | 602065 | 10p15.3 |

ADARB2 = adenosine deaminase, RNA-specific, B2 (RED2 homolog rat);
BP = base pairs;
MAF = Minor Allele Frequency

### REFERENCES

Almasy L, Blangero J (1998) Multipoint Quantitative-Trait Linkage Analysis in General Pedigrees. The American Journal of Human Genetics 62: 1198-1211
Anttila V, Kallela M, Oswell G, Kaunisto M A, Nyholt D R, Hämäläinen E, Havanka H, Ilmavirta M, Terwilliger J, Sobel E, Peltonen L, Kaprio J, Färkkilä M, Wessman M, Palotie A (2006) Trait Components Provide Tools to Dissect the Genetic Susceptibility of Migraine. The American Journal of Human Genetics 79: 85-99
Anttila V, Nyholt D R, Kallela M, Artto V, Vepsäläinen S, Jakkula. E, Wennerström A, Tikka-Kleemola P, Kaunisto M A, Hämäläinen E, Widén E, Terwilliger J, Merikangas K, Montgomery G W, Martin N G, Daly M, Kaprio J, Peltonen L, Färkkilä M, Wessman M, Palotie A (2008) Consistently Replicating Locus Linked to Migraine on 10q22-q23. 82: 1051-1063
Bass B L (2002) RNA editing by adenosine deaminases that act on RNA. Annual Review of Biochemistry 71: 817-846
Bayerer B, Engelbergs J, Savidou I, Boes T, Kiiper M, Schorn C F, Wissmann A, Knop D, Diener H C, Limmroth V (2009) Single Nucleotide Polymorphisms of the Serotonin Transporter Gene in Migraine; An Association Study. Headache: The Journal of Head and Face Pain 50: 319-322
Bellis C, Cox H, Dyer T, Charlesworth J, Begley K, Quinlan S, Lea R, Heath. S, Blangero J, Griffiths L (2008) Linkage mapping of CVD risk traits in the isolated Norfolk Island population. Human Genetics 124: 543-552
Bellis C, Hughes R M, Begley K N, Quinlan S, Lea R A, Heath S C, Blangero J, Griffiths L R (2005) Phenotypical characterisation of the isolated norfolk island population focusing on epidemiological indicators of cardiovascular disease. Hum Hered 60: 211-9
Björnsson Á, Gudmundsson G, Gudfinnsson E, Hrafnsdóttir M, Benedikz J, Skúladóttir S, Kristjánsson K, Frigge M L, Kong A, Stefánsson K, Gulcher J R (2003) Localization of a Gene for Migraine without Aura to Chromosome 441. The American Journal of Human Genetics 73: 986-993
Blangero J, Goring H H H, Kent J W, Williams J T, Peterson C P, Almasy L, Dyer T D (2005) Quantitative Trait Nucleotide Analysis Using Bayesian Model Selection. Human Biology 77: 541-559
Boerwinkle E, Chakraborty R, Sing C F (1986) The use of measured genotype information in the analysis of quantitative phenotypes in man. Annals of Human Genetics 50: 181-194
Boichard D (2002) Pedig: A fortran package for pedigree analysis suited to large populations. 7th World Congress on Genetics Applied to Livestock Production, Montpelier, France, pp. 28:13
Cader Z M, Noble-Topham S, Dyment D A, Cherny S S, Brown J D, Rice G P A, Ebers G C (2003) Significant linkage to migraine with aura on chromosome 11q24. Hum. Mol. Genet. 12: 2511-2517
Carlsson A, Forsgren L, Nylander P O, Hellman U, Forsman-Semb K, Holmgren G, Holmberg D, Holmberg M (2002) Identification of a susceptibility locus for migraine with and without aura on 6p12.2-p21.1. Neurology 59: 1804-1807
Chen C-X, Cho D-SC, Wang Q, Lai F, Carter K C, Nishikura K (2000) A third member of the RNA-specific adenosine deaminase gene family, ADAR3, contains both single- and double-stranded RNA binding domains. RNA 6: 755-767
Cologno D, Pascale A D, Manzoni G C (2003) Familial Occurrence of Migraine With Aura in a Population-Based Study. Headache: The Journal of Head and Face Pain 43:231-234
Colson N, Lea R, Quinlan S, MacMillan J, Griffiths L (2004) The estrogen receptor 1 G594A polymorphism is associated with migraine susceptibility in two independent case/control groups. neurogenetics 5: 129-133
Corominas R, Sobrido M J, Ribasés M, Cuenca-León E, Blanco-Arias P, Narberhaus B, Roig M, Leira R, López-González J, Macaya A, Cormand B (2009) Association study of the serotoninergic system in migraine in the spanish population. American Journal of Medical Genetics Part B: Neuropsychiatric Genetics 153B: 177-184
De Fusco M, Marconi R, Silvestri L, Atorino L, Rampoldi L, Morgante L, Ballabio A, Aridon P, Casari G (2003) Haploinsufficiency of ATPIA2 encoding the Na+/K+ pump [alpha]2 subunit associated with familial hemiplegic migraine type 2. Nat Genet 33: 192-196
Dichgans M, Freilinger T, Eckstein G, Babini F, Lorenz-Depiereux B, Biskup S, Ferrari MD, Herzog J, van den Maagdenberg AMJM, Pusch M, Strom T M (2005) Mutation in the neuronal voltage-gated sodium channel SCN1A in familial hemiplegic migraine. The Lancet 366: 371-377
Duggirala R, Williams J T, Williams-Blangero S, Blangero J (1997) A variance component approach to dichotomous trait linkage analysis using a threshold model. Genetic Epidemiology 14: 987-992
Dyke B (1996) PEDSYS: A pedigree data management system, 2.0 edn. Population Genetics Laboratory, Department of Genetics, Southwest Foundation for Biomedical Research, San Antonio
Fernandez F, Lea R A, Colson N J, Bellis C, Quinlan S, Griffiths L R (2006) Association between a 19 by deletion polymorphism at the dopamine beta-hydroxylase (DBH) locus and migraine with aura. Journal of the Neurological Sciences 251: 118-123
Ferrari M D, Roon K I, Lipton R B, Goadsby P J (2001) Oral triptans (serotonin 5-HT1B/ID agonists) in acute migraine treatment: a meta-analysis of 53 trials. The Lancet 358: 1668-1675
Ge D, Zhang K, Need A C, Martin O, Fellay J, Urban T J, Telenti A, Goldstein D B (2008) WGAViewer: Software for genomic annotation of whole genome association studies. Genome Research 18: 640-643
Ghosh J, Joshi G, Pradhan S, Mittal B (2010) Investigation of TNFA 308G>A and TNFB 252G>A polymorphisms in genetic susceptibility to migraine. Journal of Neurology 257: 898-904
Goadsby P J, Charbit A R, Andreou A P, Akerman S, Holland P R (2009) Neurobiology of migraine. Neuroscience 161: 327-341
Goring H H H, Terwilliger J D, Blangero J (2001) Large Upward Bias in Estimation of Locus-Specific Effects from Genomewide Scans The American Journal of Human Genetics 69: 1357-1369
Hamel E, Currents H (2007) Serotonin and Migraine: Biology and Clinical Implications. Cephalalgia 27: 1293-1300
Hanna G L, Weele JV-V, Cox N J, Van Etten M, Fischer D J, Himle J A, Bivens N C, Wu X, Roe C A, Hennessy K A, Dickel D E, Leventhal B L, Cook E H (2007) Evidence for a Susceptibility Locus on Chromosome 10p15 in Early-Onset Obsessive-Compulsive Disorder. Biological psychiatry 62: 856-862
Hoare M (1999) Norfolk Island : a revised and enlarged history 1774-1998, 5th Edition edn. Central Queensland University Press, Rockhampton
ICHD-II (2004) International classification of headache disorders, 2nd edition. Cephalalgia 24(suppl 1): 1-160
Igl W, Johansson A, Wilson J F, Wild S H, Polasek O, Hayward C, Vitart V, Hastie N, Rudan P, Gnewuch C, Schmitz G, Meitinger T, Pramstaller P P, Hicks A A, Oostra B A, van Duijn C M, Rudan I, Wright A, Campbell H, Gyllensten U (2010) Modeling of environmental effects in genome-wide association studies identifies SLC2A2 and HP as novel loci influencing serum cholesterol levels. PLoS Genetics 6(1): e1000798
Johnson M, Griffiths L (2005) A genetic analysis of serotonergic biosynthetic and metabolic enzymes in migraine using a DNA pooling approach. Journal of Human Genetics 50: 607-610
Jones K W, Ehm M G, Pericak-Vance M A, Haines J L, Boyd P R, Peroutka S J (2001) Migraine with Aura Susceptibility Locus on Chromosome 19p13 Is Distinct from the Familial Hemiplegic Migraine Locus. Genomics 78: 150-154
Juhasz G, Zsombok T, Laszik A, Gonda X, Sotonyi P, Faludi G, Bagdy G (2003) Association analysis of 5-HTTLPR variants, 5-HT2A receptor gene 102T/C polymorphism and migraine. Journal of Neurogenetics 17: 231-240
Kawahara Y, Ito K, Sun H, Aizawa H, Kanazawa I, Kwak S (2004) Glutamate receptors: RNA editing and death of motor neurons. Nature 427: 801-801
Keegan L, Leroy A, Sproul D, O'Connell M (2004) Adenosine deaminases acting on RNA (ADARs): RNA-editing enzymes. Genome Biology 5: 209
Kusumi M, Araki H, Ijiri T, Kowa H, Adachi Y, Takeshima T, Sakai F, Nakashima K (2004) Serotonin 2C receptor gene Cys23Ser polymorphism: a candidate genetic risk factor of migraine with aura in Japanese population. Acta Neurologica Scandinavica 109: 407-409
Launer L J, Terwindt G M, Ferrari M D (1999) The prevalence and characteristics of migraine in a population-based cohort: the GEM study. Neurology 53: 537-42
Lea R, Ovcaric M, Sundholm J, MacMillan J, Griffiths L (2004) The methylenetetrahydrofolate reductase gene variant C677T influences susceptibility to migraine with aura. BMC Medicine 2: 3
Lea R, Shepherd G, Curtain R, Nyholt D, Quinlan S, Brimage P, Griffiths L (2002) A typical migraine susceptibility region localizes to chromosome 1q31. neurogenetics 4: 1722
Lea R A, Ovcaric M, Sundholm J, Solyom L, Macmillan J, Griffiths L R (2005) Genetic variants of angiotensin converting enzyme and methylenetetrahydrofolate reductase may act in combination to increase migraine susceptibility. Brain Res Mol Brain Res 136: 112-7
Lipton R B, Bigal M E (2005) Migraine: Epidemiology, Impact, and Risk Factors for Progression. Headache: The Journal of Head and Face Pain 45: S3-S13
Lipton R B, Bigal M E, Diamond M, Freitag F, Reed M L, Stewart W F, on behalf of the AAG (2007) Migraine prevalence, disease burden, and the need for preventive therapy. Neurology 68: 343-349
Maas S, Kawahara Y, Tamburro K M, Nishikura K (2006) A-to-I RNA Editing and Human Disease. 2006 3: 1-9
Maas S, Rich A, Nishikura K (2003) A-to-I RNA Editing: Recent News and Residual Mysteries. Journal of Biological Chemistry 278: 1391-1394
Macgregor S, Bellis C, Lea R A, Cox H, Dyer T, Blangero J, Visscher P M, Griffiths L R (2009) Legacy of mutiny on the Bounty: founder effect and admixture on Norfolk Island. Eur J Hum Genet
Macgregor S, Bellis C, Lea R A, Cox H, Dyer T, Blangero J, Visscher P M, Griffiths L R (2010) Legacy of mutiny on the Bounty: founder effect and admixture on Norfolk Island. Eur J Hum Genet 18: 67-72
Marziniak M, Mossner R, Schmitt A, Lesch K P, Sommer C (2005) A functional serotonin transporter gene polymorphism is associated with migraine with aura. Neurology 64: 157-159
Matthews S P (2001) Norfolk Island Census of Population and Housing 7 Aug. 2001-Statistical report on characteristics of population and dwellings. Photopress International, Norfolk Island
McCarthy L C, Hosford D A, Riley J H, Bird M I, White N J, Hewett D R, Peroutka S J, Griffiths L R, Boyd P R, Lea R A, Bhatti S M, Hosking L K, Hood C M, Jones K W, Handley A R, Rallan R, Lewis K F, Yeo A J, Williams P M, Priest R C, Khan P, Donnelly C, Lumsden S M, O'Sullivan J, See C G, Smart D H, Shaw-Hawkins S, Patel J, Langrish T C, Feniuk W, Knowles R G, Thomas M, Libri V, Montgomery D S, Manasco P K, Xu C F, Dykes C, Humphrey P P, Roses A D, Purvis I J (2001) Single-nucleotide polymorphism alleles in the insulin receptor gene are associated with typical migraine. Genomics 78: 135-49
McEvoy B P, Zhao Z Z, Macgregor S, Bellis C, Lea R A, Cox H, Montgomery G W, Griffiths L R, Visscher P M (2009) European and Polynesian admixture in the Norfolk Island population. Heredity
McPeek M S, Sun L (2000) Statistical Tests for Detection of Misspecified Relationships by Use of Genome-Screen Data. The American Journal of Human Genetics 66: 1076-1094
Melcher T, Maas S, Herb A, Sprengel R, Higuchi M, Seeburg PH (1996) RED2, a Brain-specific Member of the RNA-specific Adenosine Deaminase Family. Journal of Biological Chemistry 271: 31795-31798
Meuwissen THE, Luo Z (1992) Computing inbreeding coefficients in large populations. Genetics Selection Evolution 24: 305-313.
Miller S A, Dykes D D, Polesky H F (1988) A simple salting out procedure for extracting DNA from human nucleated cells. Nucleic Acids Res 16: 1215
Mittaz L, Antonarakis S E, Higuchi M, Scott H S (1997) Localization of a novel human RNA-editing deaminase (hRED2 or ADARB2) to chromosome 10p15. Human Genetics 100: 398-400
Moskowitz M A (1992) Neurogenic versus vascular mechanisms of sumatriptan and ergot alkaloids in migraine. Trends in Pharmacological Sciences 13: 307-311
Mulder E J, van Baal C, Gaist D, Kallela M, Kaprio J, Svensson D A, Nyholt D R, Martin N G, MacGregor A J, Cherkas L F, Boomsma D I, Palotie A (2003) Genetic and Environmental Influences on Migraine: A Twin Study Across Six Countries. Twin Research 6: 422-431
Nyholt D R, Curtain R P, Griffiths L R (2000) Familial typical migraine: significant linkage and localization of a gene to Xq24-28. Human Genetics 107: 18-23
Nyholt D R, Dawkins J L, Brimage P J, Goadsby P J, Nicholson G A, Griffiths LR (1998a) Evidence for an X-linked genetic component in familial typical migraine. Hum Mol Genet 7: 459-63
Nyholt D R, Lea R A, Goadsby P J, Brimage P J, Griffiths L R (1998b) Familial typical migraine: linkage to chromosome 19p13 and evidence for genetic heterogeneity. Neurology 50: 1428-32
Nyholt D R, Morley K I, Ferreira M A R, Medland S E, Boomsma D I, Heath A C, Merikangas K R, Montgomery G W, Martin N G (2005) Genomewide Significant Linkage to Migrainous Headache on Chromosome 5q21. The American Journal of Human Genetics 77: 500-512
Ogilvie A D, Russell M B, Dhall P, Battersby S, Ulrich V, Smith C A D, Goodwin G M, Harmar A J, Olesen J (1998) Altered allelic distributions of the serotonin transporter gene in migraine without aura and migraine with aura. Cephalalgia 18: 23-26
Olesen J, Friberg L, Olsen T S, Iversen H K, Lassen N A, Andersen A R, Karle A (1990) Timing and topography of cerebral blood flow, aura, and headache during migraine attacks. Annals of Neurology 28: 791-798
Ophoff R A, Terwindt G M, Vergouwe M N, van Eijk R, Oefner P J, Hoffman S M G, Lamerdin J E, Mohrenweiser H W, Bulman D E, Ferrari M, Haan J, Lindhout D, van. Ommen G-JB, Hofker M H, Ferrari M D, Frants R R (1996) Familial Hemiplegic Migraine and Episodic Ataxia Type-2 Are Caused by Mutations in the Ca2+ Channel Gene CACNL1A4. Cell 87: 543-552
Oterino A, Castillo J, Pascual J, Cayon A, Alonso A, Ruiz-Alegria C, Valle N, Montan F, Ruiz-Lavilla N (2007) Genetic association study and meta-analysis of the HTR2C Cys23Ser polymorphism and migraine. The Journal of Headache and Pain 8: 231-235
Park J W, Han S R, Yang D W, Kim Y I, Lee K S (2006) Serotonin Transporter Protein Polymorphism and Harm Avoidance Personality in Migraine without Aura. Headache: The Journal of Head and Face Pain 46: 991-996
Paul M S, Bass B L (1998) Inosine exists in mRNA at tissue-specific levels and is most abundant in brain mRNA. EMBO J. 17: 1120-7
Peltonen L, Palotie A, Lange K (2000) Use of population isolates for mapping complex traits. Nat Rev Genet 1: 182-90
Pietrobon D, Striessnig J (2003) Neurobiology of migraine. Nat Rev Neurosci 4: 386-398
Racchi M, Leone M, Porrello E, Rigamonti A, Govoni S, Sironi G, Montomoli C, Bussone G (2004) Familial Migraine With Aura: Association Study With 5-HT1B/1D, 5-HT2C, and hSERT Polymorphisms. Headache: The Journal of Head and Face Pain 44: 311-317
Russo L, Mariotti P, Sangiorgi E, Giordano T, Ricci I, Lupi F, Chiera R, Guzzetta F, Neri G, Gurrieri F (2005) A New Susceptibility Locus for Migraine with Aura in the 15q11-q13 Genomic Region Containing Three GABA-A Receptor Genes. The American Journal of Human Genetics 76: 327-333
Schuh-Hofer S, Richter M, Geworski L, Villringer A, Israel H, Wenzel R, Munz D, Arnold G (2007) Increased serotonin transporter availability in the brainstem of migraineurs. Journal of Neurology 254: 789-796
Seeburg P H (1996) The role of RNA editing in controlling glutamate receptor channel properties. J Neurochem 66: 1-5
Seeburg P H, Higuchi M, Sprengel R (1998) RNA editing of brain glutamate receptor channels: mechanism and physiology. Brain Research Reviews 26: 217-229
Silberstein S D (2006) Preventive treatment of migraine. Trends in Pharmacological Sciences 27: 410-415
Smith H C, Gott J M, Hanson M R (1997) A guide to RNA editing. RNA 3: 1105-1123
Sobel E, Papp J C, Lange K (2002) Detection and Integration of Genotyping Errors in Statistical Genetics. The American Journal of Human Genetics 70: 496-508
Soragna D, Vettori A, Carraro G, Marchioni E, Vazza G, Bellini S, Tupler R, Savoldi F, Mostacciuolo M L (2003) A Locus for Migraine without Aura Maps on Chromosome 14q21.2-q22.3. The American Journal of Human Genetics 72: 161-167
Sprenger T, Goadsby P J (2009) Migraine pathogenesis and state of pharmacological treatment options. BMC Med. 16: 71
Stain A H, de Vries B, Janssens A C J W, Vanmolkot K R J, Aulchenko Y S, Henneman P, Oostra B A, Frants R R, van den Maagdenberg A M J M, Ferrari M D, van Duijn C M, Terwindt G M (2010) Shared genetic factors in migraine and depression: Evidence from a genetic isolate. Neurology 74: 288-294
Stewart W F, Bigal M E, Kolodner K, Dowson A, Liberman J N, Lipton R B (2006) Familial risk of migraine: Variation by proband age at onset and headache severity. Neurology 66: 344-348
Stovner L J, Hagen K, Jensen R, Katsarava Z, Lipton R B, Scher A I, Steiner T J, Zwart J A (2007) The global burden of headache: a documentation of headache prevalence and disability worldwide. Cephalalgia 27: 193-210
Svensson D A, Larsson B, Waldenlind E, Pedersen N L (2003) Shared Rearing Environment in Migraine: Results From Twins Reared Apart and Twins Reared Together. Headache: The Journal of Head and Face Pain 43: 235-244
Todt U, Freudenberg J, Goebel I, Heinze A, Heinze-Kuhn K, Rietschel M, Gobel H, Kubisch C (2006) Variation of the serotonin transporter gene SLC6A4 in the susceptibility to migraine with aura. Neurology 67: 1707-1709
Vikelis M, Mitsikostas D D (2007) The Role of Glutamate and its Receptors in Migraine. CNS &#38; Neurological Disorders—Drug Targets (Formerly Current Drug Targets—CNS &#38; Neurological Disorders) 6: 251-257
Wang Q, O'Brien P J, Chen C-X, Cho D-SC, Murray J M, Nishikura K (2000) Altered G Protein-Coupling Functions of RNA Editing Isoform and Splicing Variant Serotonin2C Receptors. Journal of Neurochemistry 74: 1290-1300
Wessman M, Kallela M, Kaunisto M A, Marttila P, Sobel E, Hartiala J, Oswell G, Leal S M, Papp J C, Hämäläinen E, Broas P, Joslyn G, Hovatta I, Hiekkalinna T, Kaprio J, Ott J, Cantor R M, Zwart J-A, Ilmavirta M, Havanka H, Färkkilä M, Peltonen L, Palotie A (2002) A Susceptibility Locus for Migraine with Aura, on Chromosome 4q24. The American Journal of Human Genetics 70: 652-662
Yilmaz M, Erdal ME, Herken H, Caataloluk O, Barlas O, Bayazit Y A (2001) Significance of serotonin transporter gene polymorphism in migraine. Journal of the Neurological Sciences 186: 27-30

1. A method for identifying a subject who has an increased risk of migraine, including the step of detecting a polymorphism in the adenosine deaminase, RNA-specific, B2 (ADARB2) gene in the nucleic acid complement of said subject, wherein the presence of said polymorphism is associated with an increased risk of migraine.

2. The method of claim 1, wherein said subject is a human.

3. The method of claim 2, wherein said human is a female.

4. The method of claim 1 further comprising detecting one or more additional polymorphisms in said ADARB2 gene, wherein the presence of said polymorphism and said one or more additional polymorphisms are associated with an increased risk of migraine.

5. The method of claim 1, wherein said polymorphism and said one or more additional polymorphisms are single nucleotide polymorphisms.

6. The method of claim 4, wherein said ADARB2 gene is human ADARB2.

7. The method of claim 6, wherein said polymorphism is a single nucleotide polymorphism (SNP) at nucleotide 1230968 of human ADARB2.

8. The method of claim 7, wherein said SNP is an adenine to guanine change at position 1230968 of human ADARB2.

9. The method of claim 8, wherein said SNP confers a threonine to alanine amino acid change in the protein encoded by human ADARB2.

10. The method of claim 6, wherein said polymorphism is a single nucleotide polymorphism (SNP) at nucleotide 1230968 of human ADARB2 and said one or more additional polymorphisms are selected from the group consisting of a SNP at nucleotide 1227868 of human ADARB2, a SNP at nucleotide 1228206 of human ADARB2 and a SNP at nucleotide 1250184 of human ADARB2.

11. A method for identifying a human subject who has an increased risk of migraine, including the step of detecting one or more polymorphisms in the adenosine deaminase, RNA-specific, B2 (ADARB2) gene in the nucleic acid complement of said subject, wherein the presence of said polymorphisms is associated with an increased risk of migraine.

12. The method of claim 11, wherein said human subject is a female subject.

13. The method of claim 11, wherein said polymorphisms are single nucleotide polymorphisms (SNPs).

14. The method of claim 13, wherein said SNPs are selected from the group consisting of a single nucleotide polymorphism (SNP) at nucleotide 1227868 of ADARB2, a SNP at nucleotide 1228206 of ADARB2, a SNP at nucleotide 1230968 of ADARB2, and a SNP at nucleotide 1250184 of ADARB2.

15. The method of claim 1, wherein migraine is migraine with aura.

16. The method of claim 1, wherein migraine is migraine without aura.

17. A kit for use in the method of claim 1, said kit comprising one or more primers, probes and one or more other reagents for identifying said polymorphism.

18. The kit of claim 17, which comprises one or more primers for nucleic acid sequence amplification of a nucleotide sequence corresponding to at least a fragment of said ADARB2 gene.

19. The method of claim 11, wherein migraine is migraine with aura.

20. The method of claim 11, wherein migraine is migraine without aura.
